(19) United States
(12) Patent Application Publication
(10) Pub. No.: US 2022/0162519 A1
(43) Pub. Date: May 26, 2022

(54) PERFUME COMPOSITION COMPRISING ESSENTIAL OIL FROM ROSEMARY AND TORREYA NUCIFERA LEAF, AND COSMETIC COMPOSITION COMPRISING SAME

(71) Applicant: UCL CO., LTD., Jeju-si (KR)

(72) Inventors: Min Jeong Kim, Busan (KR); JI Young Moon, Jeju-si (KR); Jeong Mi Kim, Jeju-si (KR); Byoung Kwon Park, Incheon (KR); A Reum Kim, Hawaseong-si (KR); Jin Hee Shin, Seoul (KR); Ji Won Lee, Seoul (KR); Jin Oh Park, Seoul (KR)

(21) Appl. No.: 17/441,005

(22) PCT Filed: May 29, 2020
(86) PCT No.: PCT/KR2020/006974

§ 371 Date: Sep. 20, 2021

(30) Foreign Application Priority Data
May 31, 2019 (KR) 10-2019-0064746

(51) Int. Cl. C11B 9/00; A61K 8/9789; A61K 8/9755; A61K 8/31; A61Q 13/00; A61Q 5/00; A61Q 19/00

(52) U.S. Cl. CPC C11B 9/0046; A61K 8/9789; A61K 8/9755; A61Q 19/007; A61Q 13/00; A61Q 5/00; A61K 8/31

(57) ABSTRACT

The present disclosure provides a fragrant material composition and cosmetic composition comprising Rosmarinus officinalis essential oil and Torreya nucifera leaf essential oil as active ingredients, wherein the Rosmarinus officinalis essential oil contains α-pinene and verbenone, and the fragrant material composition and cosmetic composition can be variously used in such fields as beauty and cosmetics by having effects of helping in stress relief and calming, tension relief, emotional stability, and mind and body stability.

### BACKGROUND OF THE DISCLOSURE

### Field of the Disclosure

The present disclosure relates to a fragrant material composition and cosmetic composition and, more particularly, to a natural fragrant material composition and cosmetic composition which provide stress relief and calming effects and help to stabilize the mind and body by comprising, as an active ingredient, a blended essential oil including Rosmarinus officinalis essential oil which contains α-pinene and verbenone, and Torreya nucifera leaf essential oil.

The present application claims priority based on Korean Application No. 10-2019-0064746 filed on May 31, 2019, and all contents disclosed in the specification of the corresponding application are incorporated in the present application by reference.

### Related Art

Stress refers to various types of displeasure or a psychologically unstable state. When stress occurs, psychological instability is caused. Due to this, a sympathetic nervous system, which is an autonomic nervous system that maintains homeostasis in the body, is overstimulated so that problems such as sleep disturbances can occur along with heightened tension. Continuous exposure to stress may cause physical diseases such as heart disease, gastric ulcers, and high blood pressure, and may also indicate psychological maladjustment such as insomnia, neurosis, or depression.

Current effect-proven stress relief methods include psychotherapy or psychical therapy, biofeedback therapy, progressive relaxation therapy, exercise therapy, or the like. Although these methods cannot directly eliminate external stressors, they are methods that allow individuals to correctly recognize various stresses which constantly come in their own lives and to live with less adverse effects on their health. Among these stress relief methods, interest in aromatherapy is increasing, and cases of using incense to provide a pleasant environment at work or public places are increasing.

In particular, fragrance substances of aroma essential oil applied to aromatherapy are known to have effects of reducing stress and maintaining the balance of the autonomic nervous system, thereby further strengthening the immunity function of controlling diseases and disharmony in the body. In some studies on aromatherapy related to stress, stress reduction effects in the work environment have also been reported through before and after subjective evaluations according to a method of inhaling some aroma oils.

### SUMMARY

The present disclosure is to solve the foregoing problems, and an object of the present disclosure is to provide a natural fragrant material composition and a cosmetic composition which provide stress relief and calming effects and help to stabilize the mind and body by comprising, as an active ingredient, a blended essential oil including Rosmarinus officinalis essential oil which contains α-pinene and verbenone, and Torreya nucifera leaf essential oil.

According to an aspect of the present disclosure, there is provided a fragrant material composition comprising Rosmarinus officinalis essential oil and Torreya nucifera leaf essential oil as active ingredients, wherein the Rosmarinus officinalis essential oil contains α-pinene and verbenone.

In the fragrant material composition, the composition may comprise 20 to 30% by weight of α-pinene and 10 to 20% by weight of verbenone with respect to the total content of the Rosmarinus officinalis essential oil.

In the fragrant material composition, the composition may comprise the Rosmarinus officinalis essential oil and Torreya nucifera leaf essential oil at a weight ratio of 5:95 to 95:5.

In the fragrant material composition, the composition may be for stress relief or mind and body stability.

According to other aspect of the present disclosure, there is provided a cosmetic composition comprising Rosmarinus officinalis essential oil and Torreya nucifera leaf essential oil as active ingredients, wherein the Rosmarinus officinalis essential oil contains α-pinene and verbenone.

In the cosmetic composition, the composition may comprise 20 to 30% by weight of α-pinene and 10 to 20% by weight of verbenone with respect to the total content of the Rosmarinus officinalis essential oil.

In the cosmetic composition, the composition may have one formulation selected from the group consisting of a toner, a lotion, a body lotion, a cream, a balm, an essence, a gel, a wax, a hair care cosmetic material, and a body care cosmetic material.

### Advantageous Effects

A fragrant material composition and cosmetic composition according to the present disclosure can be variously used in fields such as beauty and cosmetics by comprising, as an active ingredient, a blended essential oil including Rosmarinus officinalis essential oil which contains α-pinene and verbenone, and Torreya nucifera leaf essential oil, thereby having effects of helping in stress relief and calming, tension relief, emotional stability, and mind and body stability.

### DESCRIPTION OF EXEMPLARY EMBODIMENTS

Hereinafter, embodiments of the present disclosure will be described in detail. However, these embodiments are only exemplary, and the present disclosure is not limited thereto, but defined by the scope of claims to be described later.

In describing the present disclosure, when it is determined that a detailed description of a related known configuration or function may obscure the gist of the present disclosure, the detailed description will be omitted.

Terms such as first, second, A, B, (a), (b), etc. may be used to describe the present disclosure. Such terms are used for merely discriminating the constituent elements from other constituent elements, and the corresponding constituent elements are not limited in their essence, sequence, precedence, or the like by the terms.

In describing the present disclosure, when unique manufacture and material allowable errors of numerical values are suggested for mentioned meanings of terms of degrees such as “about”, “substantially”, “degree”, etc., the terms of degrees are used as the numerical values or as a meaning near the numerical values, and the terms of degrees are used to prevent an unscrupulous infringer from unfairly using a disclosure content in which exact or absolute numerical values are mentioned to help understanding of the present disclosure.

According to an embodiment of the present disclosure, there is provided a fragrant material composition comprising Rosmarinus officinalis essential oil and Torreya nucifera leaf essential oil as active ingredients, wherein the Rosmarinus officinalis essential oil contains α-pinene and verbenone.

The composition can be variously used in such fields as beauty and cosmetics by comprising a naturally-derived blended essential oil as an active ingredient, thereby having effects of helping in stress relief and calming, tension relief, emotional stability, and mind and body stability.

Rosmarinus officinalis is an evergreen wood native to the Mediterranean belonging to the family Labiatae, is one of the traditional spices in Europe which has been mainly cultivated in southern Europe, and has been used medicinally as a stomachic, a carminative, an anti-inflammatory, insecticide, etc. by containing essential oils with main ingredients such as 1,8-cineole, camphor, verbenone, α-pinene, etc., and bitter ingredients such as rosmarinic acid, etc.

The content of ingredients contained in Rosmarinus officinalis may be different depending on the growth environment, but Rosmarinus officinalis may comprise 20 to 30% by weight of α-pinene and 10 to 20% by weight of verbenone with respect to the total content of the Rosmarinus officinalis essential oil in the present disclosure.

Although the content of ingredients contained in Rosmarinus officinalis may vary depending on the growth environment, it may comprise 20 to 30% by weight of α-pinene and 10 to 20% by weight of verbenone with respect to the total content of the Rosmarinus officinalis essential oil in the present disclosure.

Although it does not mean that there is no effect such as mind and body stability, etc. at all even if α-pinene and verbenone are contained outside the above ranges, Rosmarinus officinalis is more effective in mind and body stability and brain wave stability when α-pinene and verbenone are contained in the above ranges, and it may act more effectively on stress relief and calming, tension relief, emotional stability, and mind and body stability.

Rosmarinus officinalis essential oil extracted from Rosmarinus officinalis is useful for students who need mental concentration because of its excellent effects on mental awakening, increased concentration, and memory improvement. Further, it is helpful for patients with low blood pressure since it has a cardiac effect and a blood pressure increase effect. It has excellent effects of promoting lymph circulation and relieving body fluid stasis so that it is helpful for cellulite and obesity.

Further, the Rosmarinus officinalis essential oil has an analgesic effect so that it is effective in muscle pain and joint pain, and has a wrinkle-relieving effect so that it is effective in promoting hair and preventing hair loss. Above all, it shows great efficacy in changing mood and relieving or alleviating stress by having a strong, clean, and refreshing scent. Further, it may act as a natural preservative to enable a formulated product to be safely stored.

The Rosmarinus officinalis essential oil refers to an oily extract obtained by condensing fragrance ingredients from Rosmarinus officinalis that is an extraction target. In the preparation of the essential oil extract, methods publicly-known in the art such as steam distillation, solvent extraction using volatile solvents (benzene, hexane, etc.), leaching (a method of collecting leaching sap by wounding the extraction target), supercritical extraction, etc. may be used.

The Rosmarinus officinalis essential oil may be directly obtained by using the publicly-known methods, or high-purity essential oil containing α-pinene and verbenone in the above content ranges may be purchased in the market.

Torreya nucifera is an evergreen tree that grows in the southern part of Korea and Jeju Island, and is distributed worldwide only in Korea and Japan. Torreya nucifera is used for food, ornamental, industrial, and medicinal purposes, and its seeds are eaten or used for extracting oil. Further, it has been known that its fruits are used as medicines for deworming, hair growth, strong stomach, menstruation, intestinal bleeding, etc. in oriental medicine and folk medicine, and its wood is used for building materials, equipment materials, marine materials, etc.

The Torreya nucifera leaf essential oil refers to an oily extract obtained by condensing fragrance ingredients from leaves of Torreya nucifera that is an extraction target. In the preparation of the essential oil extract, methods publicly-known in the art such as steam distillation, solvent extraction using volatile solvents (benzene, hexane, etc.), leaching (a method of collecting leaching sap by wounding the extraction target), supercritical extraction, etc. may be used.

The Torreya nucifera leaf essential oil may be directly obtained by using the publicly-known methods, or high-purity essential oil sold in the market may be purchased.

According to an embodiment of the present disclosure, there is provided a fragrant material composition or a cosmetic composition comprising the composition as an active ingredient.

The fragrant material composition or cosmetic composition may comprise the Rosmarinus officinalis essential oil and Torreya nucifera leaf essential oil at a weight ratio of 5:95 to 95:5, preferably 10:90 to 90:10, and more preferably 15:85 to 85:15. When the Rosmarinus officinalis essential oil and the Torreya nucifera leaf essential oil are mixed in the above range, the feeling of use and efficacy may be best exhibited, and when the Rosmarinus officinalis essential oil is used in an amount of less than 5 at the weight ratio, or the Torreya nucifera leaf essential oil is used in an amount of less than 5 at the weight ratio, the efficacy may not be sufficiently expressed.

The cosmetic composition may further comprise a vegetable oil, wherein the vegetable oil may be one or more selected from the group consisting of green tea seed oil, sunflower seed oil, grape seed oil, meadowfoam seed oil, soybean oil, vitamin tree oil, camellia seed oil, and citron seed oil.

The fragrant material composition or cosmetic composition may be used for stress relief or mind and body stability.

The “stress relief” refers to relieving and reducing the degree of stress, which is a non-specific biological response that occurs in the body with respect to various injuries and stimuli applied to the living body, and particularly refers to relieving mental stress or tension caused by external factors.

The “mind and body stability” may mean that the mind is comfortable and not anxious in a psychological aspect unlike physical stability, and a stable state is maintained or such a state is continued. Further, the psychological stability may mean a state in which the mind is comfortable away from stress, and it means to stabilize and relax emotions.

In an embodiment of the present disclosure, the fragrant material composition may increase alpha waves and decrease beta waves in brain waves. In the brain waves, alpha (α) waves occur in a comfortable state, beta (β) waves occur in an anxious or nervous state, theta (θ) waves occur in a happy or sleepy state, and delta (δ) waves occur during deep sleep.

As can be seen from the results of the following experimental examples, increasing the activity ratio of alpha waves by using a fragrant material composition according to the present disclosure means that the fragrant material composition has relaxing and soothing effects on the human body and allows the human body to feel comfortable.

The cosmetic composition may be a toner, a lotion, a body lotion, a cream, a balm, an essence, a gel, a wax, a hair care cosmetic material, a body care cosmetic material, etc. For example, it may be a skin lotion, a skin softener, a skin toner, an astringent, a body lotion, a milk lotion, a moisture lotion, a nourishing lotion, a massage cream, a nourishing cream, a moisture cream, a hand cream, a balm, an essence, a nourishing essence, a gel, a wax, a pack, a soap, a cleansing foam, a cleansing lotion, a cleansing cream, a body cleanser, etc.

When the cosmetic composition is used as a hair care cosmetic material, although a shampoo, a hair conditioner, a hair pack, a hair treatment, a hair soap, a hair toner, a hair lotion, a hair cream, a hair spray, a hair mousse, a hair gel, etc. can be used as the hair care cosmetic material, and preferably the shampoo or hair conditioner may be used as the hair care cosmetic material, the present disclosure is not limited thereto.

The body care cosmetic material may include bath and shower products, etc., and may be prepared in the form of a liquid soap, a solid soap, a granule, a powder, a scent bag, or a cream.

Suitable formulations of the body care cosmetic material include bath products, body shampoos, soaps, body oils, body lotions, body gels, treatment creams, skin protection ointments, shaving preparations such as shaving foams or gels, skin powders such as baby powders, moisturizing gels, moisturizing sprays, revitalizing body sprays, cellulite gels, and peeling formulations.

The cosmetic composition may be prepared in any formulation conventionally prepared in the art, and although it may be formulated into, for example, a solution, a suspension, an emulsion, a paste, a gel, a cream, a lotion, a powder, a balm, a soap, a surfactant-containing cleansing, an oil, a powder foundation, an emulsion foundation, a wax foundation, a spray, etc., the present disclosure is not limited thereto.

The cosmetic composition may comprise a conventionally acceptable carrier, and although, for example, oil, water, surfactant, humectant, lower alcohol, thickener, chelating agent, pigment, preservative, fragrance, etc. may be appropriately blended, the present disclosure is not limited thereto.

When the cosmetic composition is formulated into a powder or a spray, the cosmetic composition may comprise lactose, talc, silica, aluminum hydroxide, calcium silicate, polyamide powder, or mixtures thereof as a carrier component, and when the cosmetic composition is formulated into the spray, the cosmetic composition may further comprise a propellant such as hydrochlorofluorocarbons, propane, butane or dimethyl ether.

When the cosmetic composition is formulated into a solution or an emulsion, the cosmetic composition may comprise a solvent, a solubilizer, or an emulsifier as a carrier component. For example, the cosmetic composition may comprise water, ethanol, isopropanol, ethyl carbonate, ethyl acetate, benzyl benzoate, propylene glycol, and 1,3-butylglycol oil, and may particularly comprise cottonseed oil, peanut oil, corn germ oil, olive oil, castor oil and sesame oil, glycerol aliphatic esters, polyethylene glycol, or fatty acid esters of sorbitan.

When the cosmetic composition is formulated into a suspension, the cosmetic composition may comprise a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, or the like as a carrier component.

When the cosmetic composition is formulated into a soap, the cosmetic composition may comprise alkali metal salts of fatty acids, fatty acid hemiester salts, fatty acid protein hydrolysates, isethionate, lanolin derivatives, aliphatic alcohols, vegetable oils, glycerol, sugar, etc. as a carrier component.

The cosmetic composition may further comprise fatty substances, an organic solvent, a solubilizer, a thickening agent, a gelling agent, a softening agent, an antioxidant, a suspending agent, a stabilizing agent, a foaming agent, an air freshener, a surfactant, water, an ionic or nonionic emulsifier, a filler, a sequestering agent, a chelating agent, a preservative, a blocking agent, a wetting agent, an essential oil, a dye, a pigment, a hydrophilic or lipophilic activator which are commonly used in the industry depending on the quality or function of the final product, and supplemental agents which are commonly used in the field of cosmetology or dermatology such as any other ingredients commonly used in cosmetics.

However, the supplemental agents and their mixing ratios may be appropriately selected so as not to affect the desirable properties of a cosmetic composition according to the present disclosure.

Hereinafter, the present disclosure will be described in more detail through Examples. However, it is obvious that the present disclosure is not limited by the following Examples.

### [EXAMPLE] PREPARATION OF FRAGRANT MATERIAL COMPOSITIONS

### 1. Preparation of Rosmarinus Officinalis Essential Oil

As shown in Table 1 below, 250 g of Rosmarinus officinalis collected in each area was pulverized to a size of 3 cm. The pulverized Rosmarinus officinalis was put into a distillation essential oil extraction device, the extraction process was carried out at a temperature of 100° C. for about 4 hours to obtain Rosmarinus officinalis essential oil at a yield of 0.8 to 1.0%, contents of α-pinene and verbenone were analyzed according to the following analysis conditions, and the analysis results were written in Table 1 below.

### Analysis Conditions

The obtained Rosmarinus officinalis essential oil was analyzed using a gas chromatography-mass selective detector (GC-MS), and each analyzed peak was compared with the library. Only ingredients with a degree of congruence with the library of 95% or more were selected and determined as constitutive components.

### 1) Sample Preparation

After diluting Rosmarinus officinalis essential oil 100 times in hexane, and a resulting material obtained by filtering the diluted Rosmarinus officinalis essential oil with a 0.2 μm syringe filter was used.

| 2) Analysis equipment | |
|---|---|
| Gas Chromatography-Mass | |
| Selective Detector (Agilent 7890A) | |
| 3) Analysis conditions | |
| Flow rate: 1 ml/min | Carrier gas: helium |
| Injection Temp.: 270° C. | Oven Temp.: 40 to 250° C., 3° C./min |
| Split ratio: 20:1 | |
| 4) Column | |
| HP-5MS | |
| 5) Search library | |
| W9N08.L (Wiley) | |

TABLE 1

| | Area | α-Pinene | L-Verbenone |
|---|---|---|---|
| RM 1 | Jeju (Aewol) | 27.20% | 15.63% |
| RM 2 | Jeju (Pyoseon) | 28.54% | 9.55% |
| RM 3 | Gyeonggi-do | 28.14% | 9.53% |
| RM 4 | Gyeongsangnam-do | 25.28% | 9.89% |
| RM 5¹⁾ | Spain | 22.24% | |

¹⁾Spanish Rosmarinus officinalis essential oil purchased from Samhwa F&F

### 2. Preparation of Torreya Nucifera Leaf Essential Oil

250 g of Torreya nucifera leaves collected in Jeju area was pulverized to a size of 3 cm. The pulverized Torreya nucifera leaves were put into a distillation essential oil extraction device, the extraction process was carried out at a temperature of 100° C. for about 4 hours, and Torreya nucifera leaf essential oil was obtained at a yield of 0.2 to 0.5%.

### 3. Combinations of Fragrant Material Compositions

After homogeneously mixing the Rosmarinus officinalis essential oil extract and the Torreya nucifera leaf essential oil extract obtained by the above preparation process at a composition ratio as shown in Table 2 below, the homogeneously mixed extract was used in the test. In Table 2 below, an RM 1 essential oil was used in Examples 1 to 3, an RM 2 essential oil was used in Comparative Example 1, an RM 3 essential oil was used in Comparative Example 2, and an RM 5 essential oil was used in Comparative Example 3.

TABLE 2

| | Rosmarinus | Torreya |
|---|---|---|
| | officinalis | nucifera |
| Comparative Example | 50 | 50 |
| 1 | | |
| Comparative Example | 70 | 30 |
| 2 | | |
| Comparative Example | 20 | 80 |
| 3 | | |
| Example 1 | 50 | 50 |
| Example 2 | 70 | 30 |
| Example 3 | 20 | 80 |

### [Experimental Example] Measurement of Physiological/Psychological Effects of Fragrant Material Compositions

In order to objectively evaluate the physiological/psychological effects of the fragrant material compositions, the fragrant material compositions were provided, and electroencephalograms (EEG) and heart rates were measured. The specific method is as follows.

EEG measurements were made in a stable state in which constant temperature and humidity were maintained and noise was not mixed as much as possible. Changes in EEG of the frontal lobe were measured at 19 scalp locations using the 10-20 electrode placement method, and the frontal lobe regions (Fp1, Fp2, Fz, F3, F4, F7, F8) were analyzed. The fragrant material compositions of Table 2 above were taken, a distance of 3 cm from subjects' noses to the fragrant material compositions was maintained, and the subjects were measured for 3 minutes each before and after inhalation of incense in a state that the subjects' eyes were closed. Selected investigators did not have any special skin symptoms or unpleasant olfactory symptoms before smelling the incense, consisted of 25 adult women aged 20 to 39 years, and had no disease or drug history that could affect the test.

Further, in order to measure electrocardiogram, electrocardiogram electrodes were attached to both arms, and heart rates before and after inhaling the incense were measured.

### 1. Results of Measuring Changes in EEG

Ratios of relative α-waves to relative β-waves were analyzed by measuring central nervous responses according to the types of incense by EEG. Table 3 below shows results of analyzing the ratios of the relative α-waves to the relative β-waves according to the use of a fragrant material composition according to the present disclosure and fragrant material compositions according to Comparative Examples.

TABLE 3

| α/β | | Fp1 | Fp2 | Fz | F3 | F4 | F7 | F8 | Average |
|---|---|---|---|---|---|---|---|---|---|
| Comparative | Before | 3.561 | 3.848 | 4.449 | 3.740 | 3.897 | 3.398 | 3.493 | 3.77 |
| Example 1 | After | 3.800 | 4.417 | 4.793 | 4.599 | 4.690 | 3.796 | 3.903 | 4.29 |
| | Difference | 0.239 | 0.569 | 0.344 | 0.859 | 0.793 | 0.398 | 0.410 | 0.52 |
| | Increase or | 6.71 | 14.79 | 7.73 | 22.97 | 20.35 | 2.427 | 11.74 | 12.39 |
| | decrease rate | | | | | | | | |
| Comparative | Before | 3.555 | 3.849 | 4.688 | 3.648 | 3.901 | 3.365 | 3.186 | 3.74 |
| Example 2 | After | 4.075 | 4.383 | 5.220 | 4.172 | 4.452 | 3.686 | 3.738 | 4.25 |
| | Difference | 0.520 | 0.534 | 0.532 | 0.524 | 0.551 | 0.321 | 0.552 | 0.50 |
| | Increase or | 14.63 | 13.87 | 11.35 | 14.36 | 14.12 | 9.54 | 17.33 | 13.60 |
| | decrease rate | | | | | | | | |
| Comparative | Before | 4.016 | 3.848 | 4.824 | 3.687 | 4.006 | 3.357 | 3.355 | 3.87 |
| Example 3 | After | 4.188 | 4.417 | 4.994 | 3.978 | 4.399 | 3.474 | 3.830 | 4.18 |
| | Difference | 0.172 | 0.569 | 0.170 | 0.291 | 0.393 | 0.117 | 0.475 | 0.31 |
| | Increase or | 4.28 | 14.79 | 3.52 | 7.89 | 9.81 | 3.49 | 14.16 | 8.28 |
| | decrease rate | | | | | | | | |
| Example 1 | Before | 2.53 | 2.691 | 2.995 | 2.541 | 2.693 | 2.154 | 2.354 | 2.57 |
| | After | 3.083 | 3.005 | 3.677 | 3.1 | 3.091 | 2.447 | 2.709 | 3.02 |
| | Difference | 0.553 | 0.314 | 0.682 | 0.559 | 0.398 | 0.293 | 0.355 | 0.45 |
| | Increase or | 21.86 | 11.67 | 22.77 | 22.00 | 14.78 | 13.60 | 15.08 | 17.39 |
| | decrease rate | | | | | | | | |
| Example 2 | Before | 2.49 | 2.692 | 2.895 | 2.541 | 2.623 | 2.134 | 2.454 | 2.55 |
| | After | 3.183 | 3.105 | 3.577 | 3.142 | 3.191 | 2.427 | 2.719 | 3.05 |
| | Difference | 0.693 | 0.413 | 0.682 | 0.601 | 0.568 | 0.293 | 0.265 | 0.50 |
| | Increase or | 27.83 | 15.34 | 23.56 | 23.65 | 21.65 | 13.73 | 10.80 | 19.51 |
| | decrease rate | | | | | | | | |
| Example 3 | Before | 2.43 | 2.691 | 2.995 | 2.541 | 2.693 | 2.174 | 2.344 | 2.55 |
| | After | 2.98 | 3.075 | 3.647 | 3.121 | 3.391 | 2.447 | 2.799 | 3.07 |
| | Difference | 0.550 | 0.384 | 0.652 | 0.580 | 0.698 | 0.273 | 0.455 | 0.51 |
| | Increase or | 22.63 | 14.27 | 21.77 | 22.83 | 25.92 | 12.56 | 19.41 | 19.91 |
| | decrease rate | | | | | | | | |

Referring to Table 3 above, it can be confirmed that the α/β ratios before and after smelling the fragrant material compositions of Comparative Examples and Examples are all increased in Fp1, Fp2, Fz, F3, F4, F7 and F8. In particular, it can be confirmed that the increase or decrease rates of the α/β ratios of Comparative Examples 1 and 2 and Examples 1 to 3 in which the fragrant material compositions comprise Rosmarinus officinalis essential oil containing α-pinene and verbenone are higher than the increase or decrease rate of the α/β ratio of Comparative Examples 3 in which the fragrant material composition comprises Rosmarinus officinalis essential oil which contains α-pinene only without containing verbenone. Further, looking at Comparative Example 1 and Example 1, and Comparative Example 2 and Example 2 in which the content ratios of Rosmarinus officinalis essential oil to Torreya nucifera leaf essential oil are the same, it can be confirmed that the increase or decrease rates of the α/β ratios of Examples 1 and 2 in which the fragrant material compositions comprise Rosmarinus officinalis essential oil having a relatively high verbenone content compared to the α-pinene content.

Therefore, it can be said that a fragrant material composition according to the present disclosure comprising Rosmarinus officinalis essential oil having a relatively high verbenone content compared to the α-pinene content and Torreya nucifera leaf essential oil is shown to be more effective in mind and body stability, and it is judged that it cannot be easily predicted that the two ingredients have an effect on EEG stabilization only by simply mixing the two ingredients.

### 2. Results of Measuring Changes in Heart Rates

Changes in heart rates through electrocardiogram measurement according to incense types were analyzed, and Table 4 below shows results of analyzing changes in heart rates according to the use of fragrant material compositions.

TABLE 4

| | Comparative | Comparative | Comparative | | | |
|---|---|---|---|---|---|---|
| Heart rate | Example 1 | Example 2 | Example 3 | Example 1 | Example 2 | Example 3 |
| Before | 79.350 | 76.308 | 75.307 | 68.234 | 65.855 | 73.484 |
| After | 78.422 | 75.524 | 74.774 | 66.639 | 64.572 | 70.049 |
| Difference | −0.928 | −0.784 | −0.533 | −1.595 | −1.283 | −3.435 |
| Increase or decrease rate | −1.17 | −1.03 | −0.71 | −2.34 | −1.95 | −4.67 |
| (%) | | | | | | |

Referring to Table 4 above, it can be seen that Examples 1 to 3, which are compositions according to the present disclosure, are exhibited to have an effect on mind and body stability by significantly lowering the heart rates after smelling incense rather than before smelling incense compared to Comparative Examples 1 to 3. Looking at Comparative Example 1 and Example 1, and Comparative Example 2 and Example 2 in which the content ratios of Rosmarinus officinalis essential oil to Torreya nucifera leaf essential oil are the same, it can be seen that heart rates are more lowered in Examples 1 and 2 comprising Rosmarinus officinalis essential oil having a relatively high verbenone content compared to the α-pinene content so that Examples 1 and 2 are much more effective in stabilizing the heart rates.

### 3. Evaluation Using the Relative Anxiety Scale (STAI-X-1)

In order to evaluate the anxiety scale felt by the user before and after smelling incense, a questionnaire evaluation was conducted, 20 questions were evaluated on a 4-point scale (1. not at all, 2. somewhat, 3. moderately, 4. very much), and the evaluation means that the lower the total, the lower the anxiety.

The State-Trait Anxiety Inventory (STAI) used in the present study is a method used by Spielberger (1970) to measure the anxiety state of normal adults without mental disorders, and was adapted by Kim Jung-taek (1978).

The “State Anxiety Scale” consists of 20 questions asking how one feels about a special situation at the very moment, and the “Characteristic Anxiety Scale” consists of 20 questions asking about the innate emotional anxiety characteristics of an individual. The scoring result of each anxiety scale is structured so that the lowest 20 points to the highest 80 points can be obtained.

Table 5 below is results of analyzing the State-Trait Anxiety Inventory (STAI-X-1) according to the use of the fragrant material compositions.

TABLE 5

| | Comparative | Comparative | Comparative | | | |
|---|---|---|---|---|---|---|
| | Example 1 | Example 2 | Example 3 | Example 1 | Example 2 | Example 3 |
| Before | 35.700 | 38.100 | 37.261 | 39.56 | 39.56 | 39.56 |
| After | 33.050 | 34.500 | 33.957 | 36.11 | 35.37 | 36.21 |
| Difference | −2.65 | −3.6 | −3.304 | −3.45 | −4.19 | −3.35 |
| Increase or | −7.42 | −9.45 | −8.87 | −8.72 | −10.59 | −8.47 |
| decrease rate (%) | | | | | | |

Referring to Table 5 above, it can be seen that the compositions of Examples have lower scales of anxiety felt by users than those of Comparative Examples. Specifically, looking at Comparative Example 1 and Example 1, and Comparative Example 2 and Example 2 in which the content ratios of Rosmarinus officinalis essential oil to Torreya nucifera leaf essential oil are the same, it is confirmed that the scales of anxiety are significantly lowered in Examples 1 and 2 in which the fragrant material compositions comprise Rosmarinus officinalis essential oil having a relatively high verbenone content compared to the α-pinene content. Combining the results of Tables 3 to 5 above, a blended fragrant material composition according to the present disclosure comprising Rosmarinus officinalis essential oil having a relatively high verbenone content compared to the α-pinene content and Torreya nucifera leaf essential oil is judged to be effective in relaxation and stability of mind and body. It is judged that the blended fragrant material composition according to the present disclosure can provide psychological comfort and stability to those who suffer from discomfort due to insomnia, stress, excitement, fatigue, etc.

### Formulation Example

Cosmetics of various formulations were prepared according to the following compositions.

### 1. Lotion

As shown in Table 6 below, a lotion containing the fragrant material composition of Example 1 was prepared according to a conventional method.

TABLE 6

| Ingredient | Content (%) |
|---|---|
| Purified water | 75.3840 |
| Propylene glycol | 10.0000 |
| Butylene glycol | 2.0000 |
| Glycerin | 3.0000 |
| Lecithin | 7.0000 |
| Poloxamer 407 | 0.5000 |
| 1,2-hexanediol | 1.0000 |
| Water-soluble collagen | 0.0010 |
| Palmitoyl tripeptide-1 | 0.0100 |
| Tocopherol | 0.0500 |
| Ecklonia cava extract | 0.0500 |
| Retinyl palmitate | 0.0050 |
| Example 1 | 1.0000 |

### 2. Shampoo

As shown in Table 7 below, a shampoo containing the fragrant material composition of Example 2 was prepared according to a conventional method.

TABLE 7

| Ingredient | Content (%) |
|---|---|
| Purified water | 83.9825 |
| Sodium laureth sulfate | 5.0000 |
| Glycerin | 3.0000 |
| Sodium lauryl sulfate | 1.8000 |
| Butylene glycol | 1.5000 |
| Cocamide MEA | 1.2000 |
| Cocamidopropyl betaine | 1.1000 |
| Hydroxyethyl urea | 0.5000 |
| Sodium chloride | 0.5000 |
| Menthol | 0.3000 |
| Polyquaternium-10 | 0.3000 |
| Citric acid | 0.1800 |
| Sodium benzoate | 0.1800 |
| Potassium sorbate | 0.0750 |
| Disodium EDTA | 0.0500 |
| Sodium carbonate | 0.0300 |
| Mentha arvensis leaf extract | 0.0015 |
| Phenoxyethanol | 0.0009 |
| Ethylhexylglycerin | 0.0001 |
| Example 2 | 0.3000 |

### 3. Body Lotion

As shown in Table 8 below, a body lotion containing the fragrant material composition of Example 3 was prepared according to a conventional method.

TABLE 8

| Ingredient | Content (%) |
|---|---|
| Purified water | 76.2451 |
| Glycerin | 5.0002 |
| Cetyl ethylhexanoate | 4.0000 |
| Stearyl alcohol | 2.8000 |
| Behenyl alcohol | 2.2000 |
| Shea butter | 2.0000 |
| Glyceryl stearate | 1.1120 |
| 1,2-hexanediol | 1.0001 |
| Palmitic acid | 0.8190 |
| Stearic acid | 0.6630 |
| Cyclopentasiloxane | 0.6500 |
| Dimethicone | 0.5000 |
| Betaine | 0.5000 |
| Polysorbate 60 | 0.5000 |
| Cyclohexasiloxane | 0.3500 |
| Sorbitan stearate | 0.3000 |
| PEG-100 stearate | 0.2000 |
| Ethylhexylglycerin | 0.1500 |
| Caprylyl glycol | 0.1500 |
| Allantoin | 0.1000 |
| Carbomer | 0.1000 |
| Arginine | 0.1000 |
| Disodium EDTA | 0.0300 |
| Tocopheryl acetate | 0.0100 |
| Lauric acid | 0.0090 |
| Myristic acid | 0.0090 |
| Butylene glycol | 0.0017 |
| Ascorbic acid | 0.0010 |
| Example 3 | 0.5000 |

The above description merely exemplarily describes the present disclosure, and a person of ordinary skill in the art to which the present disclosure pertains will be able to understand that the present disclosure may be implemented in a modified form within a range that does not depart from the essential characteristics of the present disclosure. Therefore, the disclosed examples and experimental examples should be considered from an explanatory point of view rather than a limited point of view. The scope of the present disclosure is indicated in the patent claim scope rather than the foregoing description, and all differences within the scope equivalent thereto should be construed as being included in the present disclosure.

What is claimed is:

1. A fragrant material composition comprising Rosmarinus officinalis essential oil and Torreya nucifera leaf essential oil as active ingredients, wherein the Rosmarinus officinalis essential oil contains α-pinene and verbenone.

2. The fragrant material composition of claim 1, wherein the composition comprises 20 to 30% by weight of α-pinene and 10 to 20% by weight of verbenone with respect to the total content of the Rosmarinus officinalis essential oil.

3. The fragrant material composition of claim 1, wherein the composition comprises the Rosmarinus officinalis essential oil and Torreya nucifera leaf essential oil at a weight ratio of 5:95 to 95:5.

4. The fragrant material composition of claim 1, wherein the composition is for stress relief or mind and body stability.

5. A cosmetic composition comprising Rosmarinus officinalis essential oil and Torreya nucifera leaf essential oil as active ingredients, wherein the Rosmarinus officinalis essential oil contains α-pinene and verbenone.

6. The cosmetic composition of claim 5, wherein the composition comprises 20 to 30% by weight of α-pinene and 10 to 20% by weight of verbenone with respect to the total content of the Rosmarinus officinalis essential oil.

7. The cosmetic composition of claim 5, wherein the composition has one formulation selected from the group consisting of a toner, a lotion, a body lotion, a cream, a balm, an essence, a gel, a wax, a hair care cosmetic material, and a body care cosmetic material.
